(19) United States
(12) Patent Application Publication
(10) Pub. No.: US 2008/0091178 A1
(43) Pub. Date: Apr. 17, 2008

(54) Device and method for medical records storage in the eye

(76) Inventors: Murad A. Sunalp, Tulare, CA (US); Donn K. Harms, Del Mar, CA (US)

Correspondence Address: DONN K. HARMS;PATENT & TRADEMARK LAW CENTER, SUITE 100, 12702 VIA CORTINA, DEL MAR, CA, 92014, US

(21) Appl. No.: 11/546,751

(22) Filed: Oct. 11, 2006

(51) Int. Cl. A61B 18/18

(52) U.S. Cl. 606/4

(57) ABSTRACT

A method and apparatus for the storage and retrieval of information and records in the eye of an implantee or patient. Patient medical information or other data in the form of indica is imparted to an implant that is engaged into a cavity in the eye during eye surgery. The indicia imparted may be read through the clear tissue of the eye, using an optical magnifier once implanted. Indica is imparted to the implant using a laser or photographically or printing. Conventionally employed laser surgery equipment used for LASIK and similar surgeries can be easily adapted to both impart the indica to the implant and form a cavity to hold the implant for subsequent viewing of the visible indica.

### FIELD OF INVENTION

The device and method herein provide for storage of patient medical records. More particularly the disclosed device and method provide for the storage of patient medical records below the surface of the eye and out of the refractive area. The device and method are particularly well suited for implantation during a procedure for patients undergoing laser corrective eye surgery which employs a laser for vision correction.

### BACKGROUND

With the advent of computer-guided laser eye surgery, an ever larger number of patients are undergoing such procedures to correct their vision. There are many other types of eye surgeries for improving vision; however, procedures employing a laser to reshape the cornea have become a particular favorite with both patients and medical professionals. In such procedures an excimer or other laser is employed to reshape the eye's surface (cornea) to alter the way light rays enter the eye to achieve a correct focus. Laser-Assisted In Situ Keratomileusis (LASIK) treats nearsightedness, farsightedness, and astigmatism.

During a LASIK procedure the surgeon cuts a flap in the stroma, then employs an excimer laser to remove material under the flap. LASIK surgery usually takes less than one minute per eye.

Laser Epithelial Keratomileusis (LASEK) is similar to LASIK, except that the surgeon cuts a flap in the epithelium only, instead of through the epithelium and part of the stroma. he surgeon then loosens the edges of the flap with alcohol. ASEK is used mostly for people with thin or flat corneas who are poor candidates for LASIK, which requires more corneal tissue for success. Epi-LASIK is like LASEK surgery in that the flap is cut only in the epithelium, but the surgeon uses neither a blade nor alcohol. Instead, the surgeon uses an epikeratome, which is a separator that creates an epithelial sheet. Because no alcohol is involved, patients are likely to feel less pain than in alcohol procedures and will generally heal faster. Bladeless or “All Laser” LASIK is like LASIK, except that an additional laser instead of a bladed instrument (microkeratome) is used to create the flap in the front of the eye. As with regular LASIK, the flap then is lifted and laser energy is used to reshape the eye for vision correction.

With the many thousands of new patients for laser eye surgery have also come thousands of medical professionals to provide the surgery. As with any business, professionals in the medical field may change locations or may change affiliations or cease practice. Because many of the patients who undergo laser eye surgery may at some point in the future need a follow-up procedure, it is important that the records of any previous procedure be available to the subsequent operating medical professional.

However, with medical professionals retiring, changing practices or affiliations, moving, or ceasing business, it can be extremely hard for a patient to retrieve their records from a prior laser or other eye procedure. Since medicine is an art and the medical professional makes medical decisions based on all available information, it is very important that any prior laser eye procedure records be available for review in determining a course of action in a subsequent procedure.

Because of the above-noted changes in practice and other reasons for lost or unavailable medical records, a problem may be present for the subsequent eye surgeon if they cannot view the records of any prior procedures. It is especially important to know how much of the eye surface might have been removed, the size of the flaps cut in the cornea on a prior occasion, and the prior vision history before a prior correction. Without this information, the subsequent surgeon may not be able to provide the best course of action for the patient on the subsequent surgery.

Further, in addition to eyesight operations for humans, the device and method herein described and disclosed can also be employed for storage and viewable retrieval of veterinary data for pets, dogs, cats, zoo animals, or expensive agricultural animals like bulls for breeding. Animals at present are unable to talk or otherwise reasonably communicate their identity, history, ownership, or any other information about themselves. While implantable chips or swallowed device exist to store animal history and data, they require an expensive electronic scanner to discern first if they are present, and second the identification of the chip itself so the data library associated with it can be contacted.

An easily viewable device, requiring only conventional instruments for looking into the eye would be of great benefit for animals to provide an easy manner to ascertain their identity, medical and other history. Such information is an important asset to medical professionals and animal owners. Consequently, a method and apparatus for implantation of an easily read device which can be located without proprietary expensive scanners and the like and which can directly provide medical and other data regarding the recipient would be of great value and immediately widely employable.

As such, there exists an unmet need for a method and apparatus that will allow for its existence implanted in the patient to be ascertained with conventional medical equipment by direct viewing of the eye. Such a device should be capable of carrying the patient's eye surgery records for easy viewing for future review. Such a system should provide a manner to make the patient themselves the warehouse for their medical records of prior procedures. Such a device and method should be employable with humans and animals and allow for easy identification in both. Still further, such a system and method should provide the option for easy viewing of either the identity number of patient records for a lookup over a computer network, or the records themselves viewable in real time. Both should be available without the need for expensive electronic scanning equipment by allowing for viewing and identification or reproduction of prior records through the eye tissue of the cornea from a mount under the clear surface of the eye itself. Still further, such a device and method should be ascertainable for its presence in the eye, using the naked eye of an observer viewing the eye of the patient.

With respect to the above, before explaining at least one preferred embodiment of the invention in detail, it is to be understood that the invention is not limited in its application to the details of construction and to the arrangement of the components or steps set forth in the following description or illustrated in the drawings. The various apparatus and methods of the invention are capable of other embodiments and of being practiced and carried out in various ways which will be obvious to those skilled in the art once they review this disclosure. Also, the phraseology and terminology employed herein are for the purpose of description and should not be regarded as limiting.

Also, those skilled in the art will appreciate that the conception upon which this disclosure is based may be utilized as a basis for designing of other devices, methods and systems for carrying out the several purposes of the disclosed device. It is important, therefore, that the advantages and claims be regarded as including such equivalent construction and methodology in that they do not depart from the spirit and scope of this invention.

### SUMMARY OF THE INVENTION

There is disclosed and described herein, a method and apparatus to store a human patient's eye surgery or other personal or medical records within the eye of the patient. The device and method feature a small inert plastic or synthetic implant on which indica regarding or identifying records pertinent to the patient's surgical, medical, or personal history may be directly imparted by a laser or by photographic, microprinting or means. The implant would be sized for implantation into a small cavity that is formed in the cornea of the eye itself out of the viewing area of the eye. The implants can be employed in humans and in animals and in a particularly preferred embodiment would reveal their position in the eye without the aid of a magnifying means to allow the viewer to employ widely available eye viewing equipment to view the information thereon as it pertains to the person or animal in which it is implanted.

When employed to track a patient's eye surgery history, the inscription of patient data, or indicia identifying a repository for the data, can be imparted to the device by the same laser that is used for the eye surgery itself. Additionally, a cavity formed in the eye, for storage of the implant out of the viewing area of the cornea, can also be formed by the same laser during the surgery. Consequently, when used to store patient eye or other records and implanted during a laser eye surgery session, existing equipment already being used for such surgeries can be employed with software changes to impart the patient's history, or viewable history identification directly on the implant. Further, other information or identification my be imparted as desired or required.

The device and method can also be employed to hold data or record identification indica for animals or other types of human patients. When employed for data storage, the implant may have indicia written directly on the implant that may be viewed by simply using conventional eye viewing devices right through the cornea. The actual records themselves, or indicia indicating an accessible location for the records, can be read using such eye viewing instrumentation.

In a particularly preferred mode of the device and method, a material that luminesces under certain light wavelengths can be employed. This will allow the presence of the implant to be ascertained by simply shining the correct wavelength of light at the eye of the implantee, which will reveal the location of the implant by glowing. The device can then be viewed by appropriate magnification optical equipment for its information.

Subsequent to implantation and ascertaining its presence in the clear tissue of the cornea or eye and out of the field of view of the eye to avoid any impairment of vision, the implant may be easily “read” by any subsequent medical professional by simply viewing it through the surface of the eye with appropriate medical equipment like a microscope. This allows for subsequent medical professionals to ascertain the presence easily and to easily read a patient's prior history and surgery information, or ascertain a remote location using conventional equipment already available in most eye professional offices.

The indicia may be actual miniature reproductions of the indica and patient surgery chart or can be indica identifying a remote location to obtain the information. Either type of information should be easily “read” by viewing the implant through the clear eye tissue with the appropriate magnifying instrument. Such indicia can either be charts and drawings engraved on the implant itself, or numbers, letter, barcodes, or other means to provide an identification of a patient history record that may be obtained remote to the patient. One, two, and particularly three dimensional bar codes are particularly well suited to carry reproducible information since such bar codes may be encoded with data or images that are directly producible using appropriate software. Letters or numbers or one or two dimensional bar codes are well suited to identify a patient file history by an alphanumeric or other relational code, and a location identification in the form of a network address or phone number where the information relating to the identified implant, my be retrieved.

In all embodiments of the device and method, ascertaining the presence of the device itself should be easily accomplished by looking for it implanted in the cornea or other clear tissue of the eye. Looking for it may be done by using conventional eye viewing medical equipment or illuminating the eye with the proper wavelength of light and looking for a glow. Also, in all embodiments of the device, either the information itself or means for identifying a file and location of the information is directly ascertainable by viewing a surface of the implant, through the clear tissue of the eye.

In this respect, before explaining at least one embodiment of the invention in detail it is to be understood that the invention is not limited in its application to the details of construction and to the arrangement of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other methods and systems for carrying out the several purposes of the present invention. It is important, therefore, that the claims be regarded as including such equivalent construction insofar as they do not depart from the spirit and scope of the present invention.

It is an object of this invention to provide a method and apparatus for creating and mounting an implant for storage of a patient's medical records which can be placed directly into a cavity in a patient's eye, which is viewable from outside the eye.

It is a further object of this invention to provide such a method and apparatus using conventional laser and optical equipment already employed in most eye surgical centers.

It is a further object of this invention to provide such a medical and record keeping apparatus that may be read at a later date using magnification equipment that views the implant surface through the surface of the eye.

Yet an additional object of this invention is providing storage of other data and information regarding humans and animals in an outer surface of the eye viewable through that surface.

An additional object of this invention is the provision of such a record storing implant that luminesces under certain light wavelengths to reveal its presence to the naked eye.

A further object of this invention is the provision of an implant that is readable through the clear surface of the eye to reveal indicia identifying the specific implant in combination with a remote database of information pertaining to the implant identification indica.

There has thus been outlined, rather broadly, the more important features of the invention in order that the detailed description thereof may be better understood and in order that the present contribution to the art may be better appreciated. There are additional features of the invention that will be described hereinafter and which will form the subject matter of the claims appended hereto.

These together with other objects and advantages which will become subsequently apparent reside in the details of the construction and method as more fully hereinafter described and claimed, reference being had to the accompanying drawings forming a part thereof, wherein like numerals refer to like parts throughout.

### BRIEF DESCRIPTION OF THE DRAWING FIGURES

FIG. 1 depicts a conventional laser surgery wherein a flap is cut in the clear cornea and a laser employed to reshape the refractive surface of the cornea under the flap.

FIG. 2 depicts a laser imparting patient medical data, or implant identification information, to a small implant adapted to fit in a cavity or space outside the refractive surface of the cornea.

FIG. 3 shows the laser forming the cavity under the exterior surface of the eye and out of the refractive area or field of view of the eye.

FIG. 4 shows a perspective view of the implant mounted in the eye just below the exterior surface where its surface may be viewed by magnification or other equipment.

FIG. 5 depicts a typical medical chart showing the patient's prior eye surgical history and showing the size and location of any prior flaps in the eye, other medical information or data pertaining to the patient could be imparted.

FIG. 6 depicts a two-dimensional bar code which may be encoded with patient data and text which may be reproduced with proper decoding equipment once read through clear eye tissue.

FIG. 7 depicts indicia imparted to the viewable surface of the implant in the form of a bar code and alphanumeric characters.

### DETAILED DESCRIPTION OF PREFERRED EMBODIMENTS

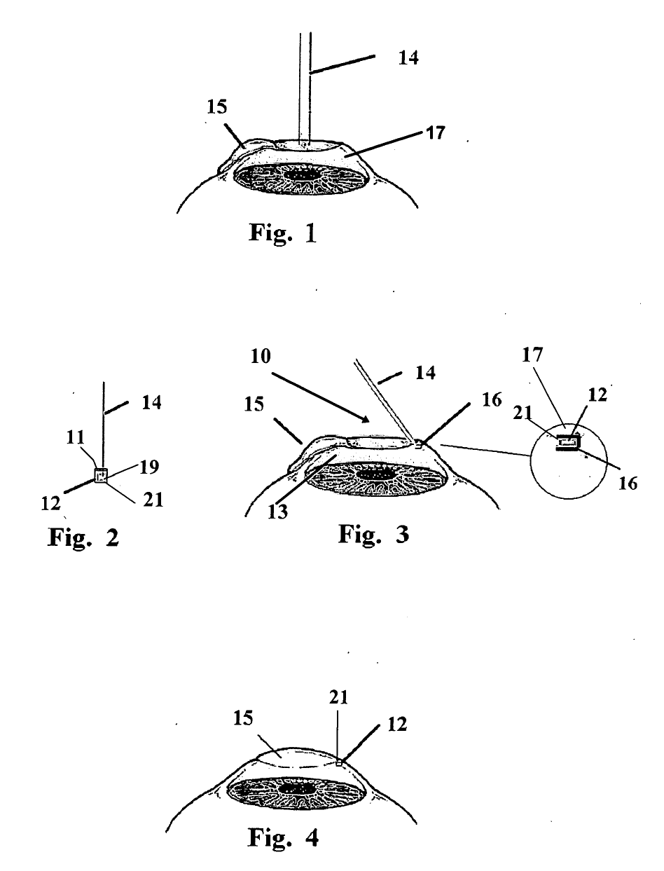

Referring now to the drawings in FIGS. 1-7, wherein similar parts are identified by like reference numerals, there is seen in FIG. 1 a perspective view of the eye during conventional laser surgery. During such a procedure, a flap 15 is cut in the cornea 17 or clear portion of the eye. The flap 15 is folded back to reveal a refractive portion of the cornea 17 in front of the lens and iris of the eye through which light is transmitted to the interior of the eye. A laser 14 is conventionally employed to reshape the refractive surface of the cornea under the flap 15 during such surgery.

Once the refractive area of the cornea 17 is reshaped, conventionally the flap 15 is replaced and heals.

In FIG. 2 there is shown one means to impart viewable indica 19 to a front surface of the implant 12. As shown, the indica 19 is imparted by writing it with a laser 14 on the viewable surface of the implant 12. The laser 12 may inscribe the indica 19 into the surface itself if the implant 12 is formed of plastic or other material adapted to that purpose or onto a prepared surface coating adapted for laser inscription formed on the viewable surface. The current preferred mode of imparting indica to the viewable surface of the implant 12 would be using the laser already employed at most medical and eye surgery centers. However, other means to impart the viewable indica 19 such as printing, photography, or other means as would occur to those skilled in the art is anticipated. The key requirement is that the indica 19 imparted to the viewable surface of the implant 12 is discernable looking through the clear eye tissue behind which it is mounted. Viewing would be by microscope or other optical equipment suitable to the task of viewing very small indica on a very small surface through the clear tissue of the eye.

The indica 19, which employed to inscribe or otherwise imparted upon the implant 12, in a particularly preferred mode would be the patient's medical data concerning the procedure being done. However, other medical data or information or images may be imparted to the viewable surface and such is anticipated since the implant may be employed as earlier noted for animals or humans for identification, tracking, or other purposes.

The indica 19 so placed would be readable through the cornea or clear surface of the eye and would be alphanumeric in nature, or images or both. One, two, and three dimensional bar codes may also be employed. The indica 19 may also be relational information which identifies the implant 12 using means to impart viewable indicia of individual identification which identifies a file of information related to the implant 12. Also included in the indica 19 may be a remote location or file server which may be contacted to retrieve the information related to the individual identified implant 12. This will allow not only viewable information about the human or animal in which the implant 12 is placed, but also means to identify a remote file of information about the implantee based on viewable indica 19 seen by the viewer through the clear tissue of the eye. Unlike medical implants requiring a scanner and radio or other receiver to identify the presence of and identity of an implant, implantation of the disclosed device 10 may be ascertained by simply looking through the clear tissue of the eye.

A particularly preferred mode of the device 10 as noted earlier, would have at least a portion 11 of the implant 12 formed of material adapted for photo luminescence when contacted by certain light spectrums such as ultra violet light. By simply shining the appropriate light spectrum in the eye, a viewing person looking through the cornea or clear tissue 17 of the implantee's eye may ascertain the presence of the device 10 without the need for magnification. Alerting to its presence without magnification will alert the professional to take further action and viewing. This component of the implant 12 would be especially helpful to any medical or other professional alerting them to the presence of a patient identifier and medical or other records that are accessible by viewing the information on the viewing surface 21. When implanted into animals or old or forgetful patients, anyone shining the appropriate light spectrum into the eye to luminance the implant 12 will be immediately informed of its presence. Veterinarians can identify the animal without the need for special wands or receivers and can thereafter read the information directly from the implant 12 to ascertain the animal's identity and/or medical history. Doctors may do the same for patients which would be especially helpful of the patient is unable to communicate.

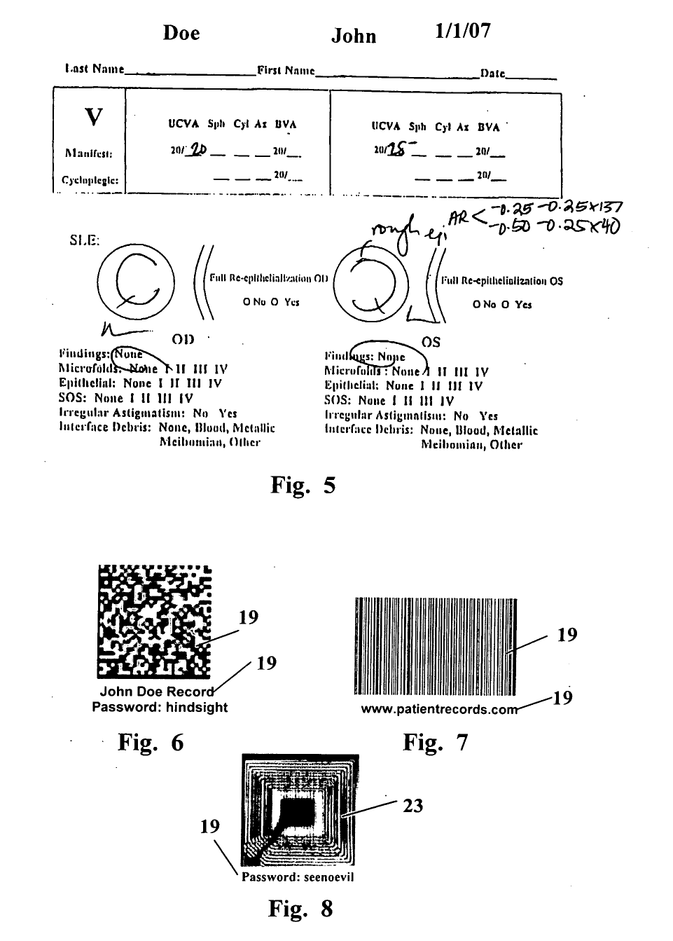

In the current preferred mode of the device 10, when implanted in eye surgery patients during laser surgery, indicia regarding the patient record data such as FIG. 5, or indicia identifying the patient and/or a remote location of patient record data and/or access codes or passwords as in FIGS. 6-8 is, in the current preferred mode, is placed upon the viewable surface 21 of the implant 12 by the laser 14 being used for the surgery itself. This can be accomplished with a simple software upgrade of existing laser eye surgery systems with some type of mounting to hold the implant 12 during inscription or impartation of the indica 19 regarding one or a combination of patient records, remote record depository, patient identification, passwords to access remotely stored data, and/or other information, onto the viewable surface of the implant 12.

The indica may be inscribed on the viewable surface 21 of the exterior surface of the implant 12 or within the interior of the implant 12. If the indicia is imparted to the interior body of the implant 12 by laser inscription, the viewable surface would need to be transparent so that the imparted indica 19 would be viewable by a person through viewable surface 21 and through the clear tissue of the cornea 17 in which it is implanted. Interior impartation of indicia, may be preferable to avoid later aberrations in viewing the indica and patient data should healing eye tissue cover the exterior of the implant 12. However, since such tissue is substantially clear like the surrounding tissue, viewing through it to the interior imparted data would work well.

As noted, other means for imparting indica 19 to the viewable surface 21 of the implant 12 are anticipated so long as such means places indica on the implant 12 which is viewable through the clear tissue of the eye using conventional optical means for magnification once the device 10 is implanted. Laser or ink printing, photographic, lithographic, or other means of imparting the indica 19 that would occur to those skilled in the art are anticipated in the scope of this patent.

In a slightly less preferred mode of the device, the implant 12 could house an RFID 23 or other passive electronic memory holding device which does not require electric power to hold electronic memory. RFID's are particularly attractive as they may be energized to transmit their stored records, using exterior power, and hold a lot of data. While the preferred mode of the device uses a laser, photographic, or some other means to impart readable indica on or into the implant 12, so it can be viewed in a low-tech manner by a simple microscope or other magnification means by any medical professional subsequently viewing into the eye tissue, RFID devices, while requiring software and communications abilities to receive the data stored, could also provide a means to store large amounts of medical or other data and information in the patient's eye for later retrieval thereby preventing complications from lost records and such.

If an RFID device is used to store electronic data, the implant 12 should provide indica 19 on the viewable surface 21 through the clear tissue or cornea 17 of the eye, that provides a key or password to allow such a communication with the RFID somewhat like as in FIG. 8. The indicia 19 may also provide some means of identification of radio frequency or other required information needed to communicate with the secured RFID. This will allow the person reading the indica 19 to ascertain the required communications protocols and passwords to receive and transmit information to the RFID. Positioning the identification information and/or other communication information on the viewable surface 21 thereby prevents unauthorized communication with the RFID and allows medical professionals or others to gain access by reading the access information through the clear tissue of the eye.

However, since imparting indica 19 is easily achieved using the same laser 14 already employed to operate upon the patient, or microprinting, or other conventional means of imprinting, and since that indica 19 would be easily read using conventional magnification means such as microscopes that are readily available in doctors' offices in industrialized nations as well as third world countries, laser or other means for directly imparting of the indica 19 to the viewable surface 21 for carrying patient record data, or photographic or imprinting means of imparting the indica 19, may be preferred as more usable in a low-tech environment and for the ability to retrieve the written information in a low-tech subsequent retrieval.

Once the human or animal patient data or other information desired for storage and easy viewing through the eye is imparted to the implant 12, or before, depending on the preference of the laser system manufacturer or whether the information will be electronically stored in addition to indicia 19, in a preferred mode of the device used during surgery, a cavity 16 is formed under the exterior cornea 17 or other clear surface area of the eye and out of the refractive field of view of the patient. The cavity 16 can be formed by the same laser 14 employed for the LASIK or similar surgery during reshaping of the cornea. The cavity 16 should as noted be formed out of the refractive area and in a current preferred mode would be located adjacent to the hinged area 13 of engagement of the flap 15. Locating the implant 12 in or adjacent to the hinged area 13 it has been determined, provides the most strength and least interference with subsequent eye surgeries if the flap 15 must be dismounted and is therefor a most preferred location; however, it is anticipated the implant 12 can be placed anywhere outside the refractive area of the cornea 17 for an operative installation such that the viewing surface 21 and or the luminescent area 11 may be seen from outside the eye.

Currently, laser equipment employed for eye surgeries is tested by focusing the laser on a test piece plastic or of other test material and uses the focused beam for test cutting. The laser also is employed to cut or shape the cornea 17 according to calculated parameters for eyesight desired. Consequently, the installed base of laser eye surgery equipment in use already can be employed with software changes to direct the laser 14 to cut eye tissue to form the properly dimensioned and positioned cavity 16 to house the implant 12. Further, placing the implant in some type of holder and directing the laser to engrave or otherwise impart the indica 19 readable from the viewable surface 21 is also achievable using current equipment with software modifications. If desirable, a separate laser might be employed adapted to the specific purpose of forming the cavity 16 during surgery and while the flap 15 is folded back, or to write the indicia 19.

As depicted in FIG. 4 the implant 12 is mounted by the surgeon in the eye, in the formed cavity 16, just below the clear tissue on the exterior eye surface, where it may be viewed easily and directly using widely available magnification equipment. Subsequent medical professionals could as such simply look into the patient's eye and read the medical records imparted to the implant 12. They could also print it out using a printer adapted for engagement to the magnification equipment. Typical actual patient data imparted to the implant 12 is shown in FIG. 5 showing the patient's eye surgical history, and showing the size and location of any prior flaps 15 in the eye would be most helpful to subsequent medical professionals. As noted, this information would be easily “read” by subsequent medical professionals using conventionally available magnification equipment employed to examine eyes. As also indicated elsewhere in the specification, other indica 19 relating directly to the implantee, or a patient identity, and/or regarding remote locations of implantee data, and/or access codes to access implantee data stored remotely, as shown in FIGS. 6-8 for example, can also be placed on the implant 12 in a manner allowing viewing from outside the eye through the cornea 17 or clear eye tissue subsequent to implantation.

For employment in the eye when laser surgery is not being performed, the device 10 can be deployed in a microsphere injection into the stroma or cornea 17 or other clear tissue outside the refractive zone or by insertion into the clear area in other manners during a lens replacement surgery or other eye surgery or even during an eye or medical exam. Injectional placement in the eye would allow the implant 12 with readable indica 19 on a viewable surface 21 to be placed in the eye at any time.

Subsequent to injection, with the proper viewing equipment noted above, the indica 19 may be directly read through the clear tissue of the cornea 17 and information ascertained as to the individual implant 12 and/or implantee and/or location of remote information related to the implant 12 identified viewing the indica 19 on the viewable surface through the clear eye tissue. Information as to access codes to access the information related to the identified patient and/or implant should also be visible to the person reading the viewable surface 21 of the implant through the cornea or clear tissue 17 of the eye. The injectable embodiment my have a broader scope of potential use in humans and animals as a means of identifying and tracking the implantee which may be initiated by viewing actual indica 19 on the implant itself subsequent to implantation.

In a method of injecting the implant 12, the indicia 19 would be imparted to a viewable surface 21 of the implant 12. The implant would be positioned in an injection device which allows for positioning of the viewable surface 21 toward outside of the eye where it can be read as shown in FIGS. 3 and 4. The device 10 would be implanted through injection into a portion of the cornea or clear tissue 17 of the eye, outside the refractive zone, with its viewable surface 21 facing the exterior of the eye. Thereafter, the indica 19 or the luminescent portion 11 would be readable through the clear eye tissue as noted above.

The device and method for storage and retrieval of patient medical records in the eye as disclosed and described herein, discloses steps in a process, arrangements of elements of particular construction, and configuration for illustrating preferred embodiments of structure and method of operation of the present invention. It is to be understood, however, that elements of different construction and configuration and different steps and process procedures and other arrangements thereof, other than those illustrated and described, may be employed for providing programming updates of interrelated electronic components with the spirit of this invention.

As such, while the present invention has been described herein with reference to particular embodiments thereof, a latitude of modifications, various changes and substitutions are intended in the foregoing disclosure, and it will be appreciated that in some instance some features of the invention could be employed without a corresponding use of other features without departing from the scope of the invention as set forth in the following claims. All such changes, alternations and modifications as would occur to those skilled in the art are considered to be within the scope of this invention as broadly defined in the appended claims.

What is claimed is:

1. A method of storage and retrieval of viewable information in the eye, comprising the steps of:
imparting indicia to an implant which may be viewed looking at a first surface thereof, said implant having an exterior surface defining a dimension;
implanting said implant in the clear tissue of the eye with said first surface facing toward the exterior of the eye and viewable through said clear tissue from a position exterior to the eye, subsequent to implantation.

2. The method of storage and retrieval of viewable information of claim 1 wherein said implanting is accomplished by the steps of:
forming a cavity in said clear tissue the eye tissue adapted to engage said dimension of said implant; and
implanting said implant in said cavity with said first surface facing toward the exterior of the eye and viewable through said clear tissue from a position exterior to the eye, subsequent to implantation.

3. The method of storage and retrieval of viewable information of claim 1 wherein said implanting is accomplished by the steps of:
microsphereal injection of the implant into a the cornea or substantially clear tissue of the eye.

4. The method of storage and retrieval of viewable information of claim 1 further comprising:
employing said magnification means, to read said indica, through said clear tissue of the eye.

5. The method of storage and retrieval of viewable information of claim 2 further comprising:
employing said magnification means, to read said indica, through said clear tissue of the eye.

6. The method of storage and retrieval of viewable information of claim 3 further comprising:
employing said magnification means, to read said indica, through said clear tissue of the eye.

7. The method of storage and retrieval of viewable information of claim 1 further comprising:
said imparting indica is provided by the step of employing means for imparting said indica to said implant from a group of means for imparting indica including a laser, printing and photography.

8. The method of storage and retrieval of viewable information of claim 2 further comprising:
said imparting indica is provided by the step of employing means for imparting said indica to said implant from a group of means for imparting indica including a laser, printing and photography

9. The method of storage and retrieval of viewable information of claim 3 further comprising:
said imparting indica is provided by the step of employing means for imparting said indica to said implant from a group of means for imparting indica including a laser, printing and photography

10. The method of storage and retrieval of viewable information of claim 4 further comprising:
said imparting indica is provided by the step of employing means for imparting said indica to said implant from a group of means for imparting indica including a laser, printing and photography.

11. The method of storage and retrieval of viewable information of claim 5 further comprising:
said imparting indica is provided by the step of employing means for imparting said indica to said implant from a group of means for imparting indica including a laser, printing and photography

12. The method of storage and retrieval of viewable information of claim 6 further comprising:
said imparting indica is provided by the step of employing means for imparting said indica to said implant from a group of means for imparting indica including a laser, printing and photography

13. The method of storage and retrieval of viewable information of claim 2 further comprising:
employing a laser to form said cavity in said eye tissue;
said cavity adapted for engagement with said dimension of said exterior surface.

14. The method of storage and retrieval of viewable information of claim 5 further comprising:
employing a laser to form said cavity, in said eye tissue;
said cavity adapted for engagement with said dimension of said exterior surface.

15. The method of storage and retrieval of viewable information of claim 8 further comprising:
employing a laser to form said cavity in said eye tissue;
said cavity adapted for engagement with said dimension of said exterior surface.

16. An apparatus for storage information in the eye comprising:
an implant having an exterior surface defining a dimension of said implant;
means for imparting indica to said implant such that it is viewable from a position adjacent to a viewable surface by the human eye using means for magnification; and
means to position said implant into a clear portion of eye tissue with said viewable surface facing an exterior of the eye, whereby said viewing surface is readable using said means for magnification from a position adjacent to the exterior surface of the eye.

17. The apparatus for storage information in the eye of claim 16 additionally comprising:
said indicia is one or a combination of a bar code and alphanumeric characters.

18. The apparatus for storage information in the eye of claim 16 additionally comprising:
said means for imparting indica to said implant includes:
a programmable RFID engaged to said implant, said RFID adapted to store information electronically and prevent access thereto without an access code;
a portion of said indica imparted to said implant and viewable while looking at said viewable surface through said clear portion of said eye; and
said portion of indicia including said access code and allow access to said information on said RFID.

19. The apparatus for storage information in the eye of claim 16 wherein said means to position said implant in the clear tissue of the eye comprises:
a laser, said laser providing means to form a cavity in said clear tissue adapted in size to accommodate said implant.

20. The apparatus for storage information in the eye of claim 18 wherein said means to position said implant in the clear tissue of the eye comprises:
a laser, said laser providing means to form a cavity in said clear tissue adapted in size to accommodate said implant.

21. The apparatus for storage information in the eye of claim 16 additionally comprising:
at least portion of said implant formed of material that luminesces under a determined light wavelength whereby a presence of said implant in an eye may be determined by an unaided eye of a person viewing said implant through said clear eye tissue when illuminated by said determined light wavelength.

22. The method of storage and retrieval of viewable information of claim 1 further comprising:
forming at least a portion of said implant from material which luminesces under a determined light wavelength.

23. The method of storage and retrieval of viewable information of claim 22 further comprising:
illuminating said eye at said determined light wavelength to ascertain if said implant luminesces thereby revealing a presence of said implant.
